(19) United States
(12) Patent Application Publication
(10) Pub. No.: US 2013/0253383 A1
(43) Pub. Date: Sep. 26, 2013

(54) GRADIENT SEQUENTIAL THERMAL COMPRESSION THERAPY APPARATUS AND SYSTEM

(71) Applicant: Maldonado Medical LLC, Phoenix, AZ (US)

(72) Inventor: Gregory Brian Maxon-Maldonado, Phoenix, AZ (US)

(73) Assignee: Maldonado Medical LLC, Phoenix, AZ (US)

(21) Appl. No.: 13/890,113

(22) Filed: May 8, 2013

Related U.S. Application Data
(63) Continuation-in-part of application No. 13/890,090, filed on May 8, 2013.
Application No. 13/890,090 is a continuation of application No. 12/501,258, filed on Jul. 10, 2009, now Pat. No. 8,485,995.
(60) Provisional application No. 61/134,677, filed on Jul. 10, 2008.
(60) Provisional application No. 61/134,676, filed on Jul. 10, 2008.

(51) Int. Cl. A61H 1/00; A61F 7/00

(52) U.S. Cl. CPC A61H 1/00; A61F 7/00
USPC 601/15

(57) ABSTRACT

A method for providing a combined DVT and sequential gradient and targeted compression therapy to a patient is provided. The method includes providing a control unit configured to condition heat transfer fluid and to selectively provide a compressed gas, providing a thermal sequential gradient compression device that is mountable to a select portion of the patient, and programming the control unit to supply heat transfer fluid to the thermal compression device and to supply compressed gas to the thermal compression device.

### CONTINUITY DATA

This application is a continuation-in-part of, claims priority to and the benefit of, U.S. Ser. No. 13/890,090 filed May 8, 2013 and entitled “Thermal Compression Therapy Apparatus and System.” The '090 application is a continuation of, claims priority to and the benefit of, U.S. Ser. No. 12/501,258 filed Jul. 10, 2009 and entitled “SYSTEM AND METHOD FOR THERMAL COMPRESSION THERAPY.” The '258 application claims priority to and the benefit of U.S. Provisional Application Ser. Nos. 61/134,676 and 61/134,677, filed on Jul. 10, 2008. All of which are incorporated herein in their entirety.

### FIELD

The disclosure relates to a medical device and therapy system. More particularly, the disclosure includes an apparatus, method and system for providing sequential gradient pressure and/or compression therapy with combined heating or cooling for use, for example, in reducing edema, pain, preventing deep vein thrombosis (DVT) and/or lymphedema.

### BACKGROUND

Various medical devices have been developed to deliver warming therapy and cooling therapy to patients recovering from injuries or surgeries. Additionally, it is known to provide a pressurized massage therapy (sometimes referred to as external pneumatic compression (“EPC”)) to these patients. Typical recipients of these therapies are patients recovering from orthopedic surgeries or injuries to various areas of the anatomy, particularly legs, knee and shoulder joints. Cooling therapy, heating therapy, and compression therapy can also be combined with a motion therapy in which a patient's joint is carefully and slowly moved through its natural motion so as to maintain flexibility in the joint. The above-described therapies have proven useful in, for example, speeding recovery and avoiding deleterious impacts of deep vein thrombosis.

Nevertheless, existing systems and methods continue to suffer from various shortcomings and are in need of improvement. One such shortcoming relates to the high cost associated with the core portion of the thermal compression therapy apparatus. The core portion, that portion of the apparatus that is wrapped around an area of the patient's body (e.g., a knee joint) is generally an expensive item. It comprises various tubings and channels that distribute the fluids through the core, so that they will surround the area to be treated. However, the core necessarily comes into contact with the patient. Thus, the core can easily become contaminated with blood and other discharges and fluids emitted by the recovering patient. Also, good hygiene practices also call for the sterilization of each core after a treatment if the core is to be reused. However, given the somewhat delicate nature of the materials and structures contained within the core, a sterilization process is not effective. Given the cost of the core, throwing each core away after a single use is an expensive option. Thus, a need exists to find a way to easily reuse cores so that a single core can be used multiple times before it needs to be discarded.

Further, the core structure itself suffers from various limitations in the present design and is in need of improvement. As previously described, a patient's joint (e.g., the knee joint) can be gradually flexed during a treatment. This movement of the joint necessarily flexes the core that is wrapped around the joint. During such therapies, there is a tendency within the core to fold and obstruct portions of the core that is repeatedly being bent. The cores can then suffer from malfunction or poor performance (even distribution of fluids) as various tubings are obstructed. There exists a need to overcome this shortcoming in current core designs.

An additional need for improvement relates to the heating and cooling therapy applied to the patient. In current methods, there is no direct way to determine the skin temperature of the patient in that area where the patient is receiving therapy. The skin is typically covered by the core. However, that is an important item of data to assure that the patient's body is not being overheated (burned) or overly chilled (frost bitten). Elderly patients or patients with severe trauma may suffer from an inability to sense temperature extremes; thus it falls upon the attending technician, nurse, or other professional to maintain a proper temperature. While the temperature exiting from the control machine can sometimes be programmed there is needed a way to confirm that the temperature at the patient also corresponds to that temperature. Hence, it would be desired to provide a core that enables a quick, easy, and reliable detection and confirmation of the patient's skin temperature where covered by the core.

### SUMMARY

The present disclosure relates to a method, apparatus and system for compression and thermal therapy that addresses, among other things, the aforementioned deficiencies in prior systems. A method, apparatus and system for sequential gradient compression and thermal therapy is presented. According to various embodiments, the method, system and/or apparatus described herein provides for multiple uses and an improved function, both with relation to fluid distribution and with respect to temperature detection. Further, the thermal compression therapy described herein may be adapted for use with present controllers and pneumatic devices. An improved therapy core configured to provide robust and strong performance, while at the same time, providing cost advantages over presently known systems and methods is described herein.

According to various embodiments, a combined heating, cooling, and sequential calibrated gradient and/or targeted compression therapy system is provided. The system may be configured for automated use with a controller. The therapy system may also be particularly designed for easy use with multiple patients. The therapy system comprises a reusable core through which a separate sequential gradient compression apparatus and a heating/cooling apparatus are positioned. The core may be placed in a disposable outer cover. The disposable cover is typically the outer portion of the system that would come into contact with a patient's body. Thus, the outer cover can comprise an impermeable layer such that blood and other bodily fluids cannot pass through the cover and contact the core. In various embodiments, the outer cover comprises an opening such that the core can be easily passed through the opening and into an interior volume of the cover, and the interior volume of the cover is designed to closely hold the core in a desired position. The outer cover, which comes into contact with the patient, can comprise a temperature nodule attached to a surface for monitoring the temperature of the patient's skin. At any suitable time, such as in response to a treatment session being completed, the core can be removed (and later reused), and the outer cover may be disposed.

According to various embodiments, an improved therapeutic system for providing a combination of compression therapy and cold and heat therapy is disclosed. The system comprises a core having a plurality of separate channels for providing cold and heat and compression therapy; a cover for receiving the core; and a skin sensitive temperature node attached to the core cover. The therapy system can further be configured such that the cover defines an interior volume and an exterior region. The cover comprises an opening such that the core can be positioned within the interior volume of the cover by passing through the opening. Further, the cover can comprise a closure system, such as hook and loop attachment system (Velcro® type fastener) disposed proximate to the opening and configured to permit the opening to be opened and closed. According to various embodiments, the temperature node measures skin temperature as by way of a thermometer or thermocouple. In various embodiments, a thermometer having a visual indicator such as a color coded thermometer is used.

Other independent features and advantages of the gradient thermal compression therapy apparatus and system will become apparent from the following detailed description, taken in conjunction with the accompanying drawings which illustrate, by way of example, the principles of described herein.

### DETAILED DESCRIPTION OF THE FIGURES

With reference to the following description, appended claims, and accompanying drawings, below is a summary of the drawing figures, wherein like numerals denote like elements and wherein:

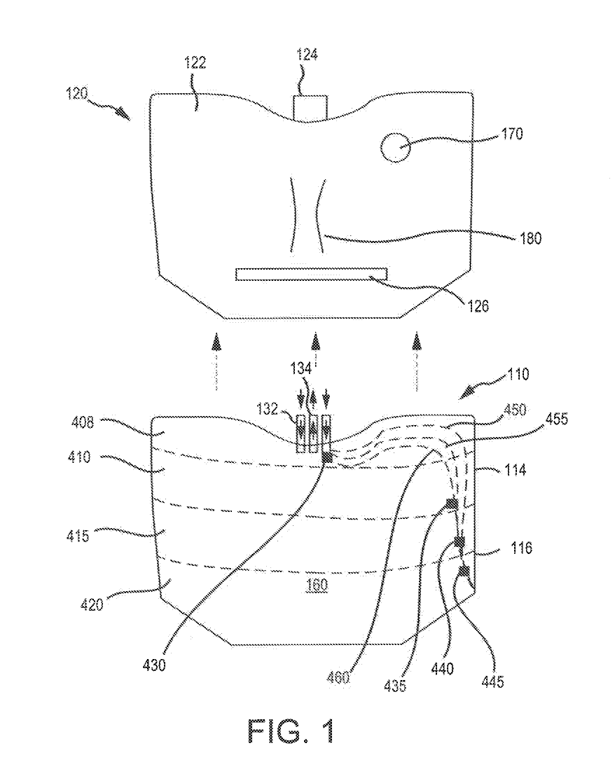

FIG. 1 is overhead plan view of a gradient thermal compression therapy unit adapted for use with a patient's appendage, showing a cover and a core configured for receipt by the cover in accordance with various embodiments;

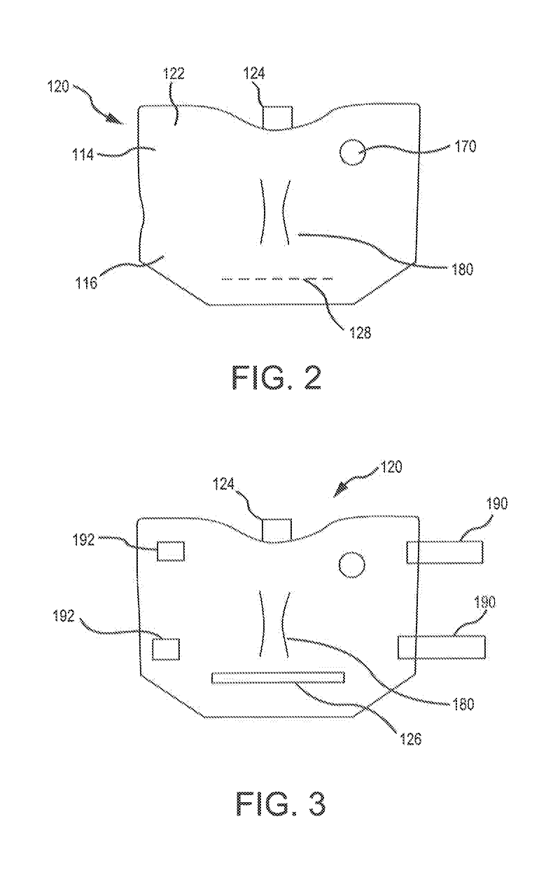

FIG. 2 is an overhead plan view of the cover of FIG. 1, illustrating a closing means in accordance with various embodiments;

FIG. 3 is an overhead plan view of the cover of FIG. 1, illustrating straps and receiving areas in accordance with various embodiments;

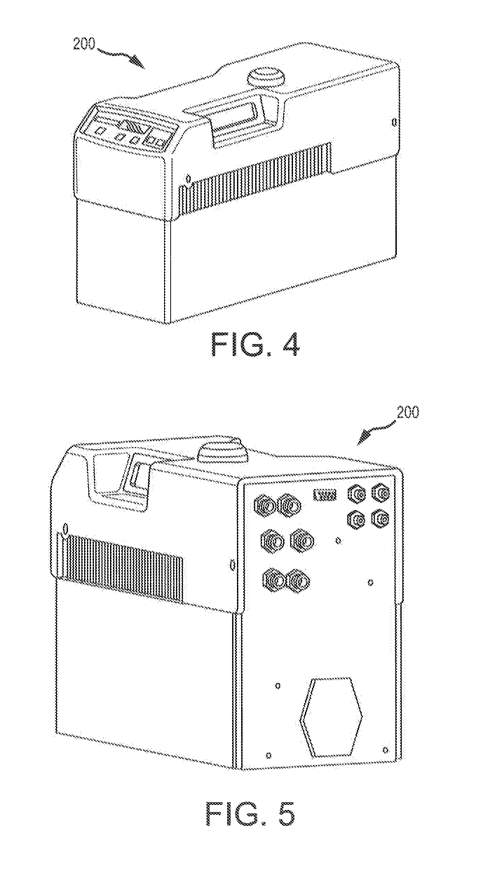

FIG. 4 is a front perspective view of a control unit in accordance with various embodiments;

FIG. 5 is a rear perspective view of the control unit of FIG. 4 in accordance with various embodiments;

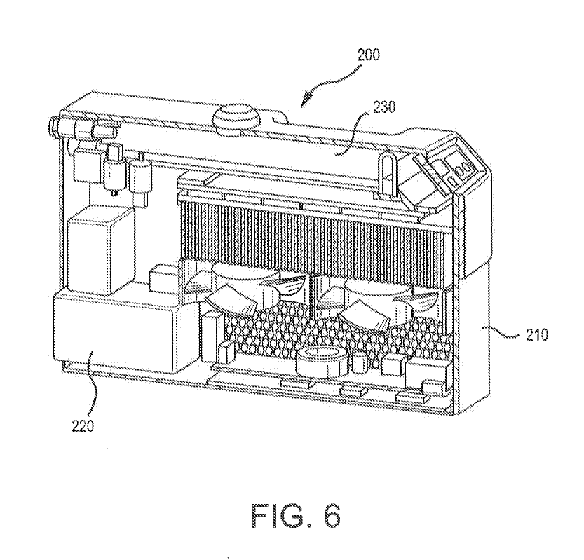

FIG. 6 is a partial cut away side perspective view of the control unit of FIG. 4, illustrating a reservoir and a power supply, in accordance with various embodiments;

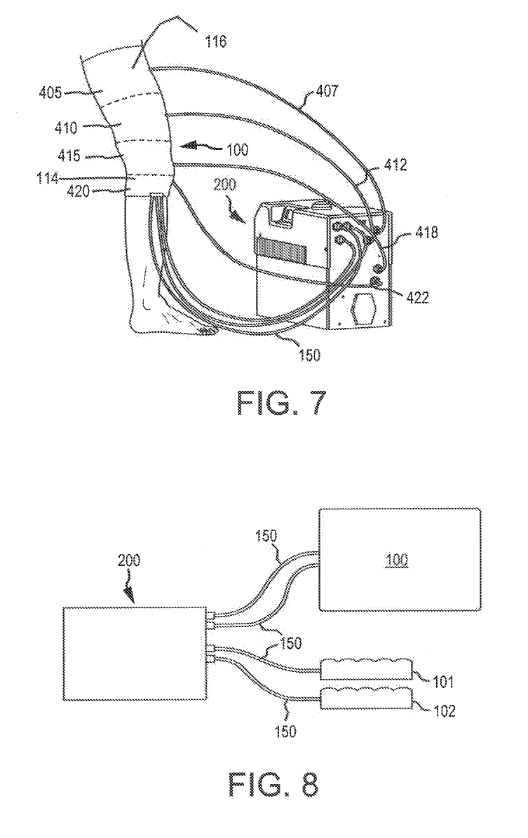

FIG. 7 is a side perspective view of a gradient thermal compression therapy system in use on a patient's thigh, in accordance with various embodiments;

FIG. 8 is a diagrammatical view of the a gradient thermal compression therapy system, illustrating various types of thermal compression units, in accordance with various embodiments;

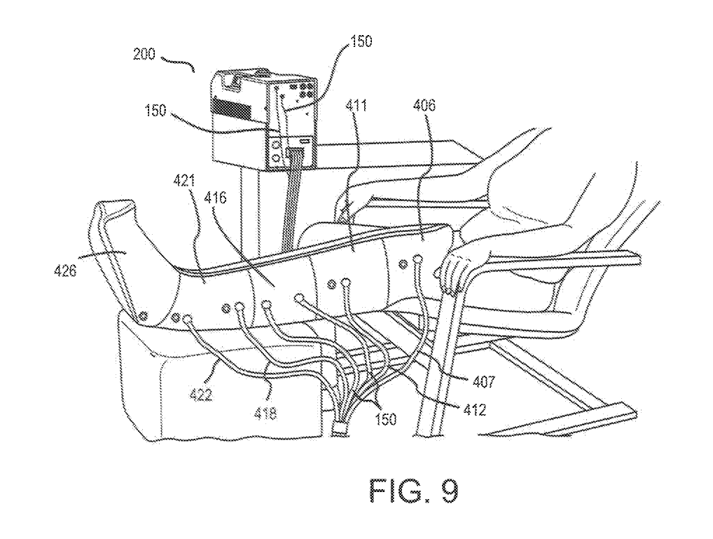

FIG. 9 is a side perspective view of a gradient thermal compression therapy system in use on a leg of a patient, in accordance with various embodiments; and

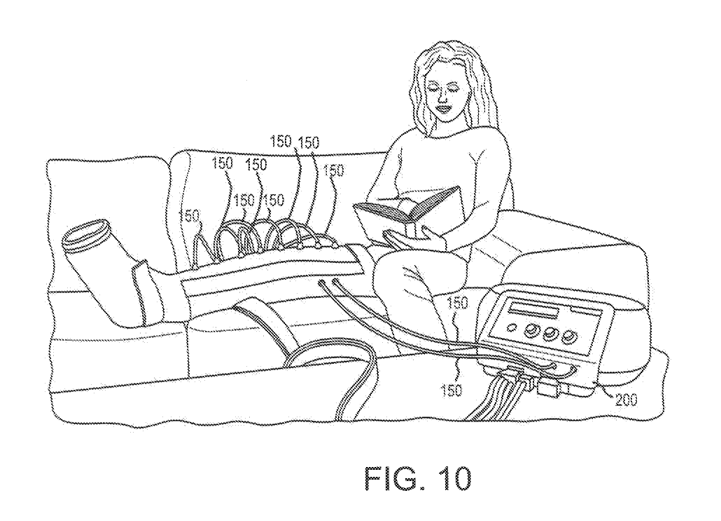

FIG. 10 is a side perspective view of a gradient thermal compression therapy system depicted on a leg of a patient, in accordance with various embodiments.

### DETAILED DESCRIPTION

The detailed description of exemplary embodiments herein makes reference to the accompanying drawings, which show exemplary embodiments by way of illustration and their best mode. While these exemplary embodiments are described in sufficient detail to enable those skilled in the art to practice the inventions, it should be understood that other embodiments may be realized and that logical, chemical and mechanical changes may be made without departing from the spirit and scope of the inventions. Thus, the detailed description herein is presented for purposes of illustration only and not of limitation.

For example, the steps recited in any of the method or process descriptions may be executed in any order and are not necessarily limited to the order presented. Furthermore, any reference to singular includes plural embodiments, and any reference to more than one component or step may include a singular embodiment or step. Thus, for example, reference to “a core” can include two or more such cores unless the context indicates otherwise. Also, any reference to attached, fixed, connected or the like may include permanent, removable, temporary, partial, full and/or any other possible attachment option. Additionally, any reference to without contact (or similar phrases) may also include reduced contact or minimal contact.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Referring now to FIG. 1, in accordance with various embodiments, a combined sequential gradient thermal compression therapy unit 100 or system is presented. The gradient thermal compression unit 100 comprises a core 110 and cover 120. While the core 110 and cover 120 are shown in an exploded arrangement in FIG. 1, in usage, the core 110 can be assembled with the cover 120 as explained further herein. The core 110 comprises at least one first channel 130 and at least one second channel 140. The channels 130, 140 can be connected by tubing 150. Generally, the cover 120 defines an interior volume 160 for receiving the core 110 and an exterior region 122. In various embodiments, the exterior region 122 of the cover 120 comprises an impermeable surface which is generally the surface of the cover 120 that comes into contact with a patient.

As would be understood by a person skilled in the art, liquid tubing 150 and gas (or air) tubing 150 can be connected to a supply machine or control unit 200 that supplies fluid such as a liquid and/or gas materials to the core 110 via the tubing 150. A In the case of liquid tubing 150, a fluid can be heated or chilled to a desired temperature. In various embodiments, the fluid comprises distilled water, and can also comprise distilled water and alcohol. This fluid is then directed through liquid tubing 150 (typically having both an inlet 350 and an outlet 355 tubing 150) so as to circulate the fluid through the channels 130, 140 and thereby to provide heating or chilling therapy to a patient. The channels 130, 140 may comprise a series of baffles or other flow directing structures to distribute the fluid to the desired zones or sections of the core 110. In various embodiments, a wide distribution of fluid is provided to avoid significant temperature gradients between one section and area of the core 110 to another. In addition, gas tubing 150 can receive a gas fluid (such as air) also directed from the supply machine or control unit 200. A separate network of channels 130 may distribute gas through the core 110. The pressure of the gas can be controlled and varied so as to provide a rhythmic pulsing of pressure around the patient's body 310 where the core 110 is positioned. Thus, for example, in the treatment of a knee, the rhythmic cycling of gas pressure increases and decreases the air pressure in the core 110 around the knee to assist with circulation among other desired benefits. Thus, it will be appreciated that the gas network of channels 130 can be separate from the liquid network of channels 130 in the core 110 so that each material provides its function; the liquid provides temperature therapy and the gas provides a pressure/compression % lymphedema treatment and/or therapy. In concert with this treatment, the patient's body 310, such as a knee, can also be receiving a motion therapy, such as rhythmically straightening and flexing the knee joint. Thus, thermal compression units may be configured so as not to interfere with any possible flexion therapy. Additionally, while the embodiments described above include providing pressure using compressed gas, it is also contemplated that other substances and/or techniques may be used to provide the desired compression.

In accordance with various embodiments and with continued reference to FIG. 1, a core 110 configured to provide sequential gradient pressure is provided. In this way, the core 110 may comprise additional chambers/zones 405, 410, 415, 420 which may be individually pressure controlled. Thus, zones 405, 410, 415, 420 may be pressure sealed relative to each other. In accordance with various embodiments, liquid for thermal treatment may travel from zone to zone as those chambers are separate from the liquid delivery chamber. Each zone 405, 410, 415, 420 may comprise its own fluid and/or air input (and associated liquid output) such that the thermal properties and compression/pressure of each zone may be individually controlled by control unit 200.

Core 110 may comprise any number of chambers/zones. For instance, 4 zones may be desirable. In other applications, such as for a larger extremity, 8 zones may be desirable. Contiguous zones may physically overlap to reduce the likelihood of localized fluid retention and/or tissue swelling at zone connection locations. Thus, the overlapping zones may form and/or approximate a multi-chambered pneumatic sleeve with overlapping cells to gently move fluid and simulate the natural movement of the body. The pressure in each zone may be individually controlled by the control unit 200. The pressure in each zone may be controlled by the control unit 200 relative to the pressure of the other zones of core 110. For example, the pressure in the core 110 may increase progressing distal to proximate the patient's core. Stated another way, for example in a gradient thermal compression therapy unit 100 applied to the leg of a patient, at a given time the pressure applied at the foot/ankle of the pressure may be greater than the pressure applied to the thigh. This may encourage fluid to travel to the trunk of the patient for processing and expelling. This may also reduce undesirable pooling of fluid.

This sequential gradient pressure may be applied in concert with thermal therapy and/or additional compression therapy such as the deep vein thrombosis (DVT) treatments/therapy described herein. For instance, zones may be combined to provide targeted compression as desired. For example, targeted compression may be used with one or more DVT compression devices 101, 102. In various embodiments, the functionality of DVT compression devices 101, 102 may be built into the single core 110. In this way, for instance, compression may be delivered to a first portion of an extremity of a patient via a first zone 420, such as a foot, and in compression not being delivered to a first portion of an extremity of a patient such as a foot compression may be delivered to a second portion of an extremity of a patient such as a thigh via a second zone 405. In this way, the first and second zones of the gradient thermal compression unit 100 may not be contiguous. Stated another way, there may be other zones, such as a third zone 415, of the gradient thermal compression unit 100 between the first zone 420 and second zone 405. The control unit 200 may adapted to automatically cycle through the following scheme: provide a pulse of compressed gas to a first zone 420 of the thermal therapy pad to achieve first desired pressure, provide a pulse of compressed gas to a third zone 415 of the thermal therapy pad to achieve second desired pressure; and provide a pulse of compressed gas to a second zone 405 of the thermal therapy pad to achieve third desired pressure. This cycle may imitate the natural flow of lymph from the distal end of a patient limb toward a trunk the patient. As used herein, a pulse may include one or more pulses. Moreover, a pulse may include any method or device for providing compressed gas to a zone.

Also, according to various embodiments, zones may be combined to produce regions of compression. For example, a first and second zone may be combined to produce a first pressure region, then according to programming and/or a timing scheme, a second zone and third zone may be combined to produce a second pressure region. These pressure regions may travel up and/or down the device 100, such as distally to proximate in the direction of the trunk of the patient.

In accordance with various embodiments and with brief reference to FIGS. 7, and 9, each zone 405, 410, 415, 420 may be fed directly by an individual fluid tube 407, 412, 418, 422 coupled to control unit 200. Thus, the control unit 200 in communication with the processor may control the pressure in each zone 405, 410, 415, 420.

In accordance with various embodiments and with renewed reference to FIG. 1, a single fluid delivery tube, such as tubing 150 coupled to a compressed air supply may deliver air to core 110. This compressed air may then be delivered to each zone 405, 410, 415, 420 via internal/external valves 430, 435, 440, 445 and/or hoses/channels coupled to each zone. For instance, dashed paths 450, 455, 460 depict exemplary paths of these channels. These valves may be pressure sensitive such that they open in response to a threshold of pressure being met. It is contemplated that valves located on or proximate to core 110 and/or zones 405, 410, 415, 420 may be in communication with control unit 200 for selective air delivery and removal such as via a single air hose 150.

In accordance with various embodiments and with reference to FIG. 1, a single fluid delivery tube, such as tubing 150 coupled to a compressed air supply may deliver air to core 110. This compressed air may then be delivered to each zone 405, 410, 415, 420 via internal/external valves and/or an internal hose 150 of decreasing or increasing bore diameter. This diameter may restrict or increase the flow of air into each zone 405, 410, 415, 420.

According to various embodiments, the gradient thermal compression therapy unit 100 may be an E0652 device and/or compatible with an E0652 device.

Referring now to FIGS. 1, 2, and 3, in accordance with various embodiments of the gradient thermal compression unit 100, the apparatus comprises a temperature module 170. In this embodiment, the temperature module 170 is a temperature sensitive structure and/or device. Temperature module 170 can, for example, may comprise a thermometer, a thermocouple connected to a read out (typically positioned on the control unit 200), and/or a visual color-coded read out attached to a temperature sensitive structure. In various embodiments, the temperature module 170 comprises both a temperature sensitive section and a display section wherein the display provides a user readable indication enabling an attending technician or professional to quickly and easily determine temperature.

In accordance with various embodiments, the temperature module 170 may be disposed on and/or coupled to the gradient thermal compression unit 100 such that a temperature-sensitive portion of the temperature module 170 is positioned proximate to the skin of a patient when the gradient thermal compression unit 100 is disposed on the patient for therapy. Thus, in various embodiments, the temperature-sensitive portion of temperature module 170 can also be positioned on the exterior surface of the cover 120 that is designed to be in contact with the patient's skin. However, as can be appreciated, a read-out section can be placed on exterior surface of the cover 120 because it is typically the surface that is visible for observation by an attendant. In various embodiments, temperature module 170 read-out section may be disposed on and/or coupled to the exterior of the core 110 and viewable via at least partially transparent window/covering of cover 120. Wires or leads or wireless communication devices may be used to connect any portion of the temperature module 170 to other devices or support structures such as a power source, a digital read out, control systems, memories, programming, alarm systems etc. The temperature of liquid deliver to the gradient thermal compression unit 100 may be automatically adjusted in response to a measurement temperature module 170. For instance, the temperature of liquid being deliver to the core 110 may be increased and/or decreased in response to a threshold being met or exceeded. This may continue until a measured temperature returns to acceptable levels. The present system 100 may be coupled to a telemedicine system for monitoring of a patient by a remote practitioner. The control system 200 may also be coupled to a telemedicine system for remote monitoring and/or adjusting by a remote practitioner. According to various embodiments, the system 100 can record the temperature of the patient's skin at various intervals.

One advantage of an apparatus having a temperature module 170 relates to the temperature module 170 providing an accurate temperature reading on the patient's skin that generally lies under the cover 120 and overall apparatus. If a temperature in a heat therapy rises too high, there is a danger of burning the patient. Conversely, if the temperature falls too low, there is a danger of frostbite. Even when these two extremes do not occur, it is desirable to determine what the temperature of the patient's skin is as opposed to just the temperature of the fluids within the core 110 as it is the actual skin temperature that is important in judging the effectiveness of a therapy.

Referring again to the Figures and with reference to FIG. 1, in accordance with various embodiments, the gradient thermal compression unit 100 may comprise a tubing cover section 124. The tubing-cover section 124 can be a unitary portion of the cover 120, for example and not meant to be limiting. A portion of the cover 120 can be designed to extend from the main body of the cover 120 to provide protection to the liquid tubing 150 and/or the gas tubing 150. In operation, when the core 110 is disposed within the interior volume 160 of the cover 120, the liquid tubing 150 and/or gas tubing 150 can extend from the interior volume 160 through one or more aperture within the cover 120 that is designed to allow the tubings 150 to extend to the exterior region 122 of the cover 120. In this way, the liquid tubing 150 and/or gas tubing 150 can make connections with supply tubings 150 that originate in the control unit 200. The tubing-cover section 124 of the cover 120 can provide an extension of material that covers and protects and/or shroud the tubings 150 from damage or contact with skin surfaces. In various embodiments, the tubing-cover section 124 of the cover 120 can comprise padding or protective material to cushion and protect the liquid tubing 150, the gas tubing 150, and/or other connecting tubings 150.

It is noted that the gradient thermal compression unit 100 can take any desired shape to conform to specified portions of the patient's anatomy. According to various embodiments, as can be seen in the figures, the illustrated shape has proven useful for treatment of the human thigh, see FIG. 7. In usage, the upper section 116 can wrap around an area generally proximate the core 110 of the body 310 while the lower section 114 can be applied to an area generally distal the core 110 of the body 310. As can be appreciated, the gradient thermal compression unit 100 can be shaped to contour almost any extremity, including, but not limited to feet, ankles, legs, arms, calf, thigh, forearm, upper arm, shoulder, hands, wrists, and the like. The gradient thermal compression unit 100 can also be configured to affix to the extremity in such a way as to permit contraction and flexion or it can be configured to restrict movement altogether.

In various embodiments, and with reference to FIG. 8, a diagrammatical view of the gradient thermal compression therapy system 100, illustrating various types of thermal compression units in accordance with an exemplary embodiment is depicted. Compression units 101, 102 may be integral to the gradient thermal compression therapy system 100 or they may be stand-alone separate elements. Compression units 101, 102 may be worn in addition to the core 110.

In various embodiments, and with reference to FIG. 9, a full leg the gradient thermal compression unit 100 embodiment is depicted. As previously states, there may be any number of zones; however, FIG. 9 depicts 5 zones 406, 411, 416, 421, 426. Each zone may be individually pressure controlled by individual fluid delivery hoses 407, 412, 418, 422, and 428. These may deliver compressed air to zones 406, 411, 416, 421, 426 respectively. There may also be hoses 150 for delivering and receiving fluid to the entire gradient thermal compression unit 100. For instance, to deliver and receive thermally conditioned liquid. In this embodiment, the gradient thermal compression unit 100 may imitate the natural flow of lymph from the distal end of a patient limb toward a trunk the patient via a sequential contiguous zone increase of pressure in each zone. Additionally, the control unit 200 may instruct two noncontiguous zones such as Zone 426 and zone 411 and/or 406 to apply DVT therapy as disclosed herein. In this way, zone 411 may be configured to act in the manner of DVT compression device 101 described herein.

In various embodiments, and with reference to FIG. 10, an alternative control unit 200 is depicted configured for sequential gradient compression and thermal therapy. This embodiment depicts additional zones as compared with the embodiment of FIG. 9.

In various embodiments the gradient thermal compression unit 100 comprises a bridge support 180 as a separate structure or comprised in either the core 110 or the cover 120. The bridge support 180 is generally positioned proximate to the area of a middle section of the core 110. As can be appreciate, folding of the core 110 can cause pinching of the channels 130, 140, which, in turn, may cause the flow of gas or fluid to be impeded. Thus, the presence of a bridge support 180 can prevent this from occurring. Additionally, the repetitive flexing of the patient's joint can tend to induce a fold in the flexible plastic material of the channels 130, 140 this fold or bend can then lead to obstruction within the channels 130, 140 just as might happen when a drinking straw is folded in half. The bridge support 180 provides a flexible but firm reinforcing material that helps prevent and/or reduce the likelihood of channels 130, 140 from being pinched or closed. Bridge support 180 tends to inhibit an obstructing fold from forming in channels 130, 140 by providing a supportive structure.

Referring again to FIGS. 1, 2, and 3 the cover 120 is described in further detail. In various embodiments, the cover 120 is constructed of a low cost material such that cover 120 can be used as a replaceable, one time use item. The cover 120 is generally configured to closely receive the core 110. Thus, the cover 120 can be a shield against contamination of the core 110 by blood or other human discharge. The cover 120 may comprise an impermeable surface, for example, that can be constructed of a material that prevents this kind of contamination of the core 110. However, the cover 120 allows the function of the core 110 to continue unimpeded by easily transmitting heating, cooling, and pressure. Thus, after a usage has taken place, the technician or nurse can throw away the cover 120 and then reuse the core 110 in a subsequent treatment.

With reference to FIG. 3 according to various embodiments, the cover 120 is shown having fastening straps 190 and receiving areas 192 attached to both the upper section 116 and the lower section 114. In various embodiments, the fastening straps 190 and receiving areas 192 can comprise a reciprocal hook and loop attachment means (e.g., VELCRO® fabric). Thus, the cover 120 can be wrapped around a portion of the patient's body 310, and then the fastening straps 190 and receiving areas 192 can be brought into contact. In this manner the cover 120 can be securely attached to the patient. The cover 120 can also be secured in this manner when the core 110 is disposed within the cover 120. The cover 120 and/or core 110 may also be securably wrapped around an appendage of the patient and secured by a zipper closure.

As has been previously mentioned, the core 110 can be assembled with the cover 120 by passing the core 110 through the opening 126 of the cover 120 and into the interior volume 160 of the cover 120. The opening 126 can comprise a closure means 128 for securing the opening 126 in a closed position to securely hold the core 110 within interior volume 160. In various embodiments, the closure means 128 comprises a hook and loop reciprocal attachment fabric such as VELCRO® fabric; however, other embodiments may use other known kinds of fasteners such as zippers, buttons, clips and the like.

In various embodiments, a control unit 200 is presented that is adapted to provide the thermally controlled fluid and compressed gas for multiple therapeutic modalities. The control unit 200 for providing these selective features can be enclosed within a single chassis design capable of providing the described modalities. In various embodiments, the control unit 200 may comprise a separate temperature control unit 200 and a pressure control unit 200, or it can comprise a single control unit 200 housing both modalities. This selective versatility provides financial and manufacturing incentives in that the simple design selectively can provide an industrial, medical, or electro-optic version that produces only thermally controlled liquid, such as co-liquid for cooling industrial equipment, in a configuration adaptable for other applications.

In accordance with various embodiments, thermal therapy can be afforded to a patient to reduce swelling and edema while, in conjunction with the DVT prophylaxis, preventing blood from pooling in lower body 310 extremities. In accordance with various embodiments thermal therapy can be afforded to a patient to reduce swelling and edema while, in conjunction with the DVT prophylaxis, preventing blood from pooling in lower body 310 extremities. This is particularly important after surgery when anesthesia has been involved. It is well known that anesthetics often tend to reduce the wall strength of veins and, if not otherwise treated, appropriate venous pumping cannot be afforded allowing for blood pooling in clots.

In accordance with various embodiments, the control unit 200 can be provided for thermal and compression therapy. The control unit 200 can be adapted to be coupled to thermal and compression elements to be applied to a patient. In this embodiments, the control unit 200 can comprise a filter 210 for filtering the compressed gas. In various embodiments, the filter 210 may be removable. In various embodiments, the filter 210 can comprise a gas-filtering substance, such as woven netting, that can be attached by VELCRO fasteners or the like outwardly of a perforated metal grate to allow for the low-pressure drawing of gas there through. This would allow cooling of components inside the control unit 200. In various embodiments, the control unit 200 can comprise one or more fans to force gas across one or more heat transfer assemblies (HTA).

In accordance with various embodiments, a HTA can be disposed beneath a fluid reservoir 230. The reservoir 230 can be configured to store liquid to be pumped into the first channel 130 via a fluid connector. In various embodiments, the fluid connector can be configured to be coupled to one or more cores 110. As can be appreciated, in various embodiments, a dual-fan arrangement can be used. The fans can, for instance, be positioned to push and/or pull gas into the interior of the control unit 200 to distribution about the electronic components so that the gas flow is both quiet and at a rate allowing initial electronic cooling and then being available to be pushed into sections of the control unit 200 where most heat dissipation is needed.

In accordance with various embodiments, a power supply 220 can be disposed internally within the control unit 200, or it can be external thereto. In various embodiments, the power supply 220 can be a 500 Watt power supply 220. In various embodiments, additional power supplies 220 can also be used to power various components. For example, in addition to a 500 Watt power supply 220, a 65 Watt power supply 220 can be used for components requiring less power. In various embodiments, the power supplies 220 are adapted to receive a plurality of inputs so the control unit 200 can be used in a plurality of countries without requiring substantial reconfiguration.

A fluid pump, for example, can also be comprised within and/or coupled to the control unit 200 for collecting fluid from a reservoir 230 that has been thermally controlled by the HTA. Thermal electric cooing devices (TEC) can also be used. In various embodiments, the TECs a positioned between a heat sink and a thermal transfer plate in a manner to provide the requisite thermal control of the fluid within the reservoir 230.

In accordance with various embodiments, the control unit 200 for providing these selective features can be enclosed within a single chassis design capable of providing the described modalities. This selective versatility provides financial and manufacturing incentives in that the simple design selectively can provide an industrial, medical, or electro-optic version that produces only thermally controlled liquid, such as co-liquid for cooling industrial equipment, in a configuration adaptable for other applications. In various embodiments, the size of the reservoir 230 has been reduced relative to a number of earlier models of thermoelectric cooler (TEC) systems such that only around 175 Watts can be needed compared to 205 Watts for typical earlier systems. As such, the control unit 200 can be configurable with TEC assemblies maximizing efficiency. With regard to a medical modality, thermal therapy can be afforded to a patient to reduce swelling and edema while, in conjunction with the DVT prophylaxis, preventing blood from pooling in lower body 310 extremities. This is particularly important after surgery when anesthesia has been involved. It is well known that anesthetics often tend to reduce the wall strength of veins and, if not otherwise treated, appropriate venous pumping may not be afforded allowing for blood pooling in clots.

In accordance with various embodiments, a plurality of gas connectors and fluid connectors can be used to provide thermally conditioned heat-transfer fluid to a plurality of gradient thermal therapy units 100 and to provide pressurized gas to a plurality of compression therapy devices and DVT compression devices 101, 102. In various embodiments, fluid connectors are provided in to facilitate circulation of fluid in a closed loop in an outward bound and an inward bound flow of fluid to and from the fluid reservoir 230 for thermal control. In various embodiments, a single compression therapy device can be coupled to a plurality of gas connectors and the control unit 200 can be programmed accordingly to provide compressed gas in a sequenced manner to a plurality of cores 110 in the compression therapy device. For example, a first core 110 can be inflated, followed by the inflation of a second core 110, which is then followed by the inflation of a third core 110, and so on. The first core 110 can be deflated before or after the second core 112 is inflated, or the first core 110 can remain inflated until all the cores 110 are inflated.

In accordance with various embodiments, the system 100 comprises a coupler 300 configured to selectively connect the first channel 130 to the liquid source and the second channel 140 to the compressed gas source. In various embodiments, the coupler 300 comprises a body defining a first interior pathway 320 and a second interior pathway 330. Each pathway has an inlet 350 and an outlet 355. The inlet 350 for the first interior pathway 320 can be configured to selectively couple with the first channel 130, the outlet 355 of the first interior pathway 320 can be configured to selectively couple with the liquid source, the inlet 360 of the second interior pathway 330 can be configured to selectively couple with the second channel 140, and the outlet 365 of the second interior pathway 330 can be configured to selectively couple with the compressed gas source.

In accordance with various embodiments, the body of the coupler 300 further defines a third interior pathway 340 having an inlet 370 and an outlet 375. In various embodiments, the inlet 350 of the first interior pathway 320 can be configured to selectively couple with a first port 132 of the first channel 130 and the inlet 370 of the third interior pathway 330 can be configured to selectively couple with a second port 134 of the first channel 130. Additionally, the outlet 375 of the third interior pathway 340 can be configured to selectively couple with the liquid source.

As one skilled in the art will appreciate, some or all of the inlets can comprise a male or female quick disconnect coupling. Likewise, the respective outlets can comprise a complimentary quick disconnect coupling. In one exemplary embodiment, the inlets for the first interior pathway 320 and the third interior pathway 330 comprise a female quick disconnect coupling, and the outlets for the first interior pathway 320 comprise a male quick disconnect coupling. Similarly, inlet 350 for the second interior pathway 330 comprises a male quick disconnect coupling, and the outlet 355 for the second interior pathway 330 comprises a female quick disconnect coupling.

In accordance with various embodiments, the cores 110 are pressurized in accordance with the medical modality described herein and the parameters are set by the programming within the control boards of the control unit 200. Additionally, an RS232 connector for data communication with the control unit 200 may be provided. Other connections can be used such as, for example, a USB connection, or a wireless connection.

In accordance with various embodiments, the control unit 200 can be used to initiate and control different sequencing patterns, times, and pressures, depending on the type of treatment desired. In various embodiments a plurality of parameters may be specified by a user, such as, for example, the inflated pressure, the deflated pressure, the rate of inflation, the inflation-hold time, and the cycle time. For example, in one treatment modality, the control unit 200 can provide compressed gas to inflate a DVT compression device 100, 101, 102 for 3-20 seconds when the DVT compression device 100 is disposed on a calf. Thus, the control unit 200 can be configured to provide a series of timed pulses of compressed gas. The time period of the pulse can be more or less depending on the part of the body 310 being treated. For example, a pulse width of around 0.3 seconds can be desirable for a foot. Similarly, the inflation times may vary depending on whether DVT compression devices (101, 102) located on both right and left extremities are being inflated simultaneously or whether the inflation is being alternated between the devices 101, 102. For example, an inflation period of 18 seconds may be desirable for simultaneous inflation whereas an inflation period of 8 seconds may be desirable when the inflation is being alternated. Similarly, when DVT compression devices 101, 102 are disposed around a patient's right and left feet, in some situations it may be desirable to have a wide pulse width on the order of 9 seconds whereas in other situations it may be desirable to have a narrow pulse width on the order of 0.3 seconds. In addition, it may be desirable to vary the cycle times in between DVT pulses. For example, a cycle time of 20 seconds in between DVT pulses may be desirable. Similarly, it may be desirable to completely deflate the DVT compression devices 100, 101, 102 in between inflations while in other embodiments; it may be desirable to keep the DVT compression devices 100, 101, 102 partially inflated. As can be seen from the above examples, it would be desirable to have a programmable control unit 200 that can be adapted to provide DVT compression at user-specified parameters.

In accordance with various embodiments, a method for providing a combined DVT and compression therapy to a patient is disclosed. The method comprises the steps of providing a control unit 200 configured to condition heat transfer fluid and to selectively provide a compressed gas; providing a thermal compression device that is mountable to a select portion of the patient; and programming the control unit 200 to supply heat transfer fluid to the gradient thermal compression unit 100 within a first predetermined temperature range and to supply compressed gas to the thermal compression device within a first predetermined pressure range. In various embodiments, the gradient thermal compression unit 100 is in operative communication with the control unit 200.

As described in the various treatment modalities herein, the control unit 200 can be programmed to supply the heat transfer fluid and the compressed gas to the gradient thermal compression unit 100 in various manners. In fact, the control unit 200 can supply more than one gradient thermal compression unit 100. In various embodiments, the heat transfer fluid and the compressed gas are supplied to the gradient thermal compression unit 100 sequentially. In various embodiments, the sequential supply of the heat transfer fluid and the compressed gas is repeated for a predetermined number of treatment applications. In various embodiments, the supply of the heat transfer fluid at least partially overlaps in time sequence with the supply of the compressed gas. As need for the therapy regimen, the heat transfer fluid and the compressed gas can also be supplied to the gradient thermal compression unit 100 substantially simultaneously. The gradient thermal compression unit 100 can be adapted to the patient in multiple modalities regulated by the control unit 200.

As described herein, the control unit 200 can provide thermal conditioning of the heat transfer fluid to both cool and heat the thermal transfer fluid, depending on the therapy desired. In various embodiments, the heat transfer fluid is conditioned to a temperature of between about 37° F. and about 105° F. In a similar manner, the control unit 200 can provide compressed gas at pressures necessary for the particular therapy desired. In various embodiments, the control unit 200 can provide compressed gas to the gradient thermal compression unit 100 at a pressure at or below about 120 mm Hg. In various other exemplary embodiments, the control unit 200 can provide compressed gas at pressure at or below 100 mm Hg, at or below 80 mm Hg, at or below 60 mm Hg, at or below 40 mm Hg, or at or below 35 mm Hg. In various embodiments, the control unit 200 can provide compressed gas at a pressure of about 25 mm Hg.

In accordance with various embodiments, the control unit 200 can be programmed to instruct the gradient thermal compression unit 100 to movement of the pressure in the zones to stimulate the flow of the excess lymph out of the affected limb as if were flowing following the movements of the muscles. In response to the lymphatic system being prepared, such as by simple lymph drainage, self-massage and/or automated massage via aspects of gradient thermal compression unit 100 such as compression and pressure, this fluid will flow into the lymphatic vessels and eventually be returned to the bloodstream. The controller 200 may be configured according to a treatment plan. For instance, gradient thermal compression unit 100 may be configured to perform light massage to intense therapy with levels of granularity in between as desired. Not all zones may be utilized during a treatment session if desired.

In accordance with various embodiments, gradient thermal compression unit 100 may be configured for unilateral and/or bilateral use. The cycle times may be any suitable sequential gradient cycle time, such as about a 10 second, 15 second, 20 second, 25 second, time zone inflate time and about a 10 second, 5 second, 3 second zone deflate time. The pressure range of the gradient thermal compression unit 100 may be any suitable therapy range. For instance, it may be between about 0-120 mmHg.

In accordance with various embodiments, the control unit 200 can be programmed to supply the heat transfer fluid to the gradient thermal compression unit 100 for a predetermined heating time, depending upon the therapy desired. In various embodiments, the predetermined heating time is between about 5 minutes and about 25 minutes. In various embodiments, the control unit 200 can be programmed to supply the compressed gas to the gradient thermal compression unit 100 for a predetermined compression time. As can be appreciated, the method can also comprise varying the temperature of heat transfer fluid supplied to the gradient thermal compression unit 100 and/or varying the pressure of compressed gas provided to the gradient thermal compression unit 100. In various embodiments, the method can comprise supplying compressed gas to the gradient thermal compression unit 100 until it reaches a predetermined pressure and, then, allowing the gradient thermal compression unit 100 to deflate and repeating, as necessary. In various embodiments, the predetermined pressure is 35 mm Hg.

In accordance with various embodiments, the method described herein can comprise using the gradient thermal compression unit 100 on one or more body 310 parts of the patient. For example, and not meant to be limiting, the method can comprise using the gradient thermal compression unit 100 on a knee of the patient, a calf of a patient, a foot of a patient, and the like.

As described herein, the method can also comprise using a plurality of gradient thermal compression units 100, as needed and/or desired for therapy. As such, the control unit 200 can be programmed to supply heat transfer fluid to the second gradient thermal compression unit 100 within a second predetermined temperature range and to supply compressed gas to the second thermal compression device within a second predetermined pressure range. In various embodiments, the first predetermined temperature range can be substantially the same as the second predetermined temperature range. In various embodiments, the first predetermined pressure range can be substantially the same as the second predetermined pressure range. As one can appreciate, the pressures and temperatures of the first and second thermal compression devices 100, 101, 102 can also be varied.

In accordance with various embodiments, the gradient thermal compression unit 100 for a DVT and compression therapy system 100 comprises a control unit 200 comprising a processing system 100 as described herein. Specifically, the processing system 100 of the control unit 200 can comprise a memory, configured for storing a software program, a first predetermined temperature range, a first predetermined pressure range, and at least one application protocol, and a processor, coupled to the memory.

In accordance with various embodiments, the processor can be configured for executing the software program, selectively directing the supply of a heat transfer fluid at a temperature selected from the first predetermined temperature range, selectively directing the supply of a compressed gas at a pressure selected from the first predetermined pressure range; and selectively directing the supply of the heat transfer fluid and the compressed gas in accordance with an application protocol selected from the at least one application protocol. In various embodiments, the processor can be configured for directing the steps of the methods described herein.

In the detailed description herein, references to “various embodiments”, “one embodiment”, “an embodiment”, “in one aspect”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the inventions. The scope of the inventions is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Furthermore, no element, component, or method step in the present disclosure is intended to be dedicated to the public regardless of whether the element, component, or method step is explicitly recited in the claims. As used herein, the term adjacent may mean in close proximity to, but does not necessarily require contact. No claim element herein is to be construed under the provisions of 35 U.S.C. 112, sixth paragraph, unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

1. A system of gradient sequential compression therapy, the system comprising:
a control unit adapted to thermally condition a heat-transfer liquid to an adjustable temperature level and to provide compressed gas at one or more selectable pressures to one or more zones of a thermal therapy pad;
the thermal therapy pad adapted for the flow of the heat-transfer liquid there-through and being coupled to the control unit; and
the thermal therapy pad further comprising a bladder comprising a first zone and a second zone of individual pressure control,
wherein the bladder is coupled to the control unit for receiving at least a portion of the compressed gas via a first pulse to inflate the first zone of the bladder to compress the thermal therapy pad against a first portion of a first extremity of the patient;
wherein the bladder is coupled to the control unit for receiving at least a portion of the compressed gas via a second pulse to inflate the second zone of the bladder to compress the thermal therapy pad against a second portion of a first extremity of the patient.

2. The system of claim 1, further comprising a second DVT compression device coupled to the control unit and adapted to receive a third pulse of compressed gas from the control unit, the second DVT compression device adapted for securement to and compression of a portion of a second extremity of the patient.

3. The system of claim 1, wherein the control unit automatically restricts transmitting the first pulse, in response to the compressed gas being applied to a second portion of a first extremity of the patient.

4. The system of claim 2, wherein the first pulse of compressed gas is provided for a user-specified length of time and the second pulse of compressed gas is provided for a user-specified length of time.

5. The system of claim 2, wherein the control unit is adapted to provide the compressed gas to the first zone the second zone and the second DVT compression device at one or more user-specified pressures.

6. The system of claim 1, wherein the control unit is adapted to automatically cycle through the following:
provide a pulse of compressed gas to the first zone of the thermal therapy pad to achieve first desired pressure;
provide a pulse of compressed gas to a third zone of the thermal therapy pad to achieve second desired pressure; and
provide a pulse of compressed gas to the second zone of the thermal therapy pad to achieve third desired pressure.

7. The system of claim 6, wherein the cycle imitates the natural flow of lymph from the distal end of a patient limb toward a trunk the patient.

8. The system of claim 1, wherein the compressed gas provided to the thermal therapy pad is varied based on monitoring of conditions of the patient.

9. The system of claim 1, wherein the heat transfer fluid is heated from about 49° F. to about 105° F. and applied to a skin area of the patient.

10. The system of claim 1, wherein the heat transfer fluid is cooled from about 105° F. to about 49° F. and applied to a skin area of the patient.

11. The system of claim 1, wherein the heat transfer fluid is cooled from an ambient temperature of about 77° F. to a temperature of about 37° F. within a 90 minute period.

12. The system of claim 1, wherein the control unit is adapted to provide the compressed gas at a pressure in the range of about 0 to about 120 mm Hg.

13. A thermal therapy pad comprising:
a channel for receiving a thermally conditioned liquid; and
a bladder comprising a plurality of zones of discrete pressure control for receiving compressed gas, wherein a control unit is configured to thermally condition the liquid to an adjustable temperature level and to provide compressed gas at one or more selectable pressures to each zone of the plurality of zones via one or more pulses, wherein each zone of the plurality of zones is adapted to compress the thermal therapy pad against a portion of a first extremity in response to receiving the one or more pulses, wherein the control unit automatically restricts transmitting the pulses to non-contiguous zones.

14. The thermal therapy pad of claim 13, wherein a DVT compression device is coupled to the control unit and adapted to receive a second pulse of compressed gas from the control unit, the second DVT compression device adapted for securement to and compression of a portion of a second extremity of the patient.

15. The thermal therapy pad of claim 13, wherein the control unit is adapted to cycle through the following:
provide a pulse of compressed gas to a first zone of the thermal therapy pad to achieve first desired pressure;
provide a pulse of compressed gas to a second zone of the thermal therapy pad to achieve second desired pressure; and
provide a pulse of compressed gas to a third zone of the thermal therapy pad to achieve third desired pressure.

16. The thermal therapy pad of claim 13, further comprising a removable outer cover comprising an impermeable layer.

17. The thermal therapy pad of claim 16, further comprising a temperature indicator coupled to the removable outer cover.

18. A method for addressing aspects of deep vein thrombosis (DVT) in a patient utilizing compression applied to the patient, the method comprising:
automatically coordinating, by a control unit, distribution of compressed gas to a thermal compression device having a plurality of non-contiguous compression zones such that one non-contiguous compression zone is compressed at a time and the compression alternates between the non-contiguous compression zones; and
automatically coordinating, by the control unit, distribution of a heat transfer fluid to the thermal compression device,
wherein the thermal compression device is secured to the patient, and
wherein the plurality of non-contiguous compression zones of the thermal compression device are adapted to receive at least a portion of the compressed gas from the control unit.

19. A method of claim 18, wherein the thermal compression device is a sequential gradient thermal compression device.

20. A method of claim 18, wherein a removable outer cover comprising an impermeable layer cloaks the thermal compression device.
